(19) United States
(12) Patent Application Publication
(10) Pub. No.: US 2007/0134324 A1
(43) Pub. Date: Jun. 14, 2007

(54) THERAPEUTIC COMBINATIONS

(76) Inventor: Jallal Messadek, Liege (BE)

Correspondence Address: HOVEY WILLIAMS LLP, 2405 GRAND BLVD., SUITE 400, KANSAS CITY, MO, 64108, US

(21) Appl. No.: 11/625,448

(22) Filed: Jan. 22, 2007

Related U.S. Application Data
(63) Continuation-in-part of application No. PCT/BE05/00112, filed on Jul. 13, 2005.
(63) Continuation-in-part of application No. PCT/BE06/00137, filed on Dec. 22, 2006.

(30) Foreign Application Priority Data
Jul. 22, 2004 (BE) BE2004/0364

(51) Int. Cl. A61K 9/20; A61K 31/4415; A61K 31/192

(52) U.S. Cl. 424/464; 514/350; 514/571

(57) ABSTRACT

The goal of the present invention is a pharmaceutical composition including a betaine and an anti-cholesterol agent. The association and co-administration of at least a betaine allows to reducing side effects related to anti-cholesterol agents administration, in particular their deleterious effects on liver, pancreas and kidneys.

This application is a continuation in part application of the International Application No. PCT/BE2005/000112 filed on Jul. 13, 2005, and published on Jan. 26, 2006 under number WO2006007671 and claiming the priority of Belgian Patent Application BE2004/0364 filed on Jul. 22, 2004, the disclosures of which are incorporated herein by reference. This application is also a continuation in part application of the International Application No. PCT/BE2006/000137 filed on 22 Dec. 2006 and not yet published, the disclosure of which is incorporated herein by reference.

### BACKGROUND OF THE INVENTION

1. Field of the Invention

The present invention concerns pharmaceutical compositions which include betaine and an anti-cholesterol agent in a therapeutic combination.

2. Description of the Prior Art

The publications “Westphal et al, Effects of fenofibrate and gemfibrozil on plasma homocysteine Lancet. 2001 Jul. 7; 358 (9275):39-40” and “Gotto et al, Risks and benefits of continued aggressive statin therapy Clin Cardiol. 2003 April; 26(4 Suppl 3): III3-12” show that the administration of anti-cholesterol substances induces a rise in transaminases or a rise in homocysteine levels. A high level of homocysteine is a recognized factor of vascular diseases.

Publications “Ethanol-induced hepatotoxicity and protective effect of betaine. Kanbak et al Cell Biochem Funct 2001 December; 19(4):281-5” and “Betaine, ethanol, and the liver: a review. Barak. Et al, Alcohol 1996 Jul-Aug; 13(4): 395-8” show that hepatoprotection effect of betaine in animal models of experimental steatosis is reached only at very high oral amounts (0.5% of betaine unit of weight of the body, which is equivalent to 5 grams/kilo of live weight or 350 g for a man of 70 kg) but which could correspond to the severity of the pathological challenge used.

The metabolic syndrome is defined according to Lindblad et al. in the publication “Metabolic syndrome and ischemic heart disease in elderly men and women. Am J Epidemiol 2001; 153: 481-9” as the prevalence of at least 3 of the following symptoms: obesity, hypertension, raised levels of triglycerides, low HDL cholesterol level, high levels of glucose at fast.

The oral use of betaine to reduce homocysteine levels in human is known for about three decades and is abundantly documented.

DE 19910682 teaches the association of betaine with fibrates.

M. F. McCarty in Medical Hypotheses, (2000) 55(3), 189-194 describes the association of a betaine with a niacine in equimolar ratio in order to avoid hepatotoxic effects of niacines. This use of betaine is not in a controlled release form.

One knows from the present inventor's disclosure in WO 00/51596 the antithrombotic activity of betaine.

The publication “Betaine supplementation decreases plasma homocysteine concentrations but does not affect body weight, body composition, or resting energy expenditure in human subjects Ursula Schwab et al, Am J Clin Nutr 2002; 76:961” describes betaine effect, in obese volunteers, on diastolic pressure, homocysteine levels and HDL levels.

The publication “Betaine, a Promising New Agent for Patients With Nonalcoholic Steatohepatitis: Results of a Pilot Study Manal F. Abdelmalek et al, The American Journal Of Gastroenterology Vol. 96, No. 9, 2001” describes betaine hepatoprotection effect and its regulating effect on transaminases.

One knows from the present inventor's disclosure in WO 02/062322 of the controlled releases forms of betaine as well as betaine association with statins and ciprofibrate. This document does not teach the combination of a controlled release form of betaine with a form containing statins or ciprofibrate.

WO 02/062322 describes betaine association in its immediate release form with statins and ciprofibrate but does not describe a pharmaceutical composition comprising a controlled and/or delayed and/or floating release form of betaine combined with an anti-cholesterol agent.

None of the preceding publications describes a pharmaceutical composition comprising a betaine in a controlled release form and/or delayed and/or floating combined with an anti-cholesterol agent.

Betaine administration permits to lower homocysteine concentration in blood. About half of homocystinuric patients are treated with high oral doses of betaine of 6 grams to 20 grams per day. These high doses of betaine are necessary in order to reach plasmatic concentrations of about 200 to 400 μMol/L among patients and consequently to reduce homocysteine levels (An indirect response model of homocysteine suppression by betaine: optimizing the dosage regimen of betaine in homocystinuria, Angela Matthews et al, Br J Clin Pharmacol, 54, 140-146). For a patient such amounts are unacceptable in point of view of “compliance”.

It also appears that after oral administration of Cystadane® (anhydrous betaine powder) betaine absolute bioavaibility seems to be very weak, about 10% (Schwahn B C et al: Pharmacokinetics of oral betaine in healthy subjects and patients with homocystinuria. Br J Clin Pharm. 2003 January; 55 (1):6-13). In this same publication, it appears that betaine after administration quickly reaches a plasmatic peak (optimal effectiveness) of which the duration is relatively short.

In the study “Betaine, a Promising New Agent for Patients With Nonalcoholic Steatohepatitis: Results of a Pilot Study. Abdelmalek et al”, betaine is administered at oral doses of 20 grams day, i.e. to reach therapeutically effective dose in this type of pathology, similar to those induced by the anti-cholesterol agents, it is necessary to administer big amounts of betaine. Such high doses are concordant with those used in animal experiments.

It thus appears that to be able to reach a useful threshold of hepatic protection or a sufficient homocysteine reduction in the body, it is necessary to administer, in combination with the anti-cholesterol agents, big amounts of betaine detrimentally of treatment convenience, which can prove to be constraining for long course treatments and compromising patient strict compliance and hence the final therapeutic result.

For the reasons above, equimolar administration of niacines with betaine would also suffer from this lack of convenience in regard of the therapeutic amount of niacines to be administrated, i.e. 4 to 8 grams in three takes per day, for such a dose it would be necessary to add the same amount of betaine, which would be equivalent to a medicinal combination of 8 to 16 grams per day. Moreover, in light of the doses used in Abdelmalek et al. study, it is not sure that such equimolar amount is sufficient to reach betaine hepatoprotection effect.

For the same reasons of convenience, the use of delayed niacines at dose of 1000 to 2000 mg/day would be still constraining (dose of 2 to 4 grams of the combination delayed niacin/betaine with immediate release) and would get only low levels of 100 to 200 mg of total circulating betaine, which is insufficient for the required protective effect.

Concerning the association of a betaine with a fibrate the authors of DE 19910682 describe the use of up to 20 grams/day of a betaine with immediate release. In example 3 they describe an effervescent tablet containing 2 grams of betaine (with immediate release) plus 1.2 grams of Gemfibrozil, plus the excipients allowing effervescence, which according to the state of the art in galenic should give an effervescent tablet with ±4 to 5 grams total weight and more than 2 centimeters diameter. Such a drug would not be very convenient for a long term use, its size and presentation being likely to compromise a strict observance of its use. Moreover, the amount of betaine used risk to not get the desired protection either on the liver or in homocysteine reduction.

Concerning the combination of a betaine with statins in WO 02/062322, the inventor describes a combination where betaine is in immediate release form, i.e. to reach a protective effect it is necessary to administer several grams per day. The statins are administered at very low dose (0.2 mg to 80 mg per day) and a combination with a betaine with immediate release in sufficient amount for hepatoprotection and/or therapeutic effect (i.e. several grams) could compromise the optimal absorption of these statins due to their very small ratio in the aforementioned combination. In fact, in such a combination the statins would be drowned and carried away by the excess of betaine, this when reducing the amount of statins which would reach blood flow would compromise their therapeutic effectiveness. A combination in this form, betaine being a surfactant, would have the disadvantage to wash or to mask the very tiny therapeutic amount of administered statins, hence the need for a particular administration of betaine in order to avoid losing or masking the anti-cholesterol substance.

It thus appears that the combinations of immediate release betaine with anti-cholesterol agents previously described present numerous disadvantages. These disadvantages are due notably to possible interactions of betaines with anti-cholesterol agents on absorption sites, to the large amounts of betaine to be administered before reaching therapeutic concentrations for protective purpose which do not seem be reached in these former combinations, and to the lack of control of betaines presence and concentration in the body. Moreover, previous combinations describe constraining galenic forms lacking convenience of use which is a proven factor of bad observance in long course treatments and which would compromise the final therapeutic result.

### SUMMARY OF THE INVENTION

Surprisingly, the inventor discovered that the fact of combining a betaine with controlled and/or delay and/or floating release form with an anti-cholesterol agent permits to avoid such disadvantages. The combinations of the invention improve convenience of use as treatments' observance, are surer and more effective, and such result being reached while reducing the amount of betaine and even the amount of anti-cholesterol agent to be administered. By controlling optimally betaine presence and concentration in the body, the combinations of the invention, in regard to betaine numerous pharmacological properties, are therapeutically more effective and surer compared to former combinations as to existing treatments. The use of controlled and/or delayed and/or floating releases forms of betaine in the combinations of the invention constitutes, by the advantages which it brings, a very notable improvement of the state of art.

The goal of the present invention is a pharmaceutical composition comprising a betaine and an anti-cholesterol agent. The association and co-administration of at least a betaine allows to reducing side effects related to anti-cholesterol agents' administration, notably their deleterious effects on liver, pancreas and kidneys. Anti-cholesterol agents' administration also induces elevation in liver enzymes or transaminases and/or elevation in homocysteine levels in the body which the association and the co-administration of at least a betaine permit to reduce. From its cardiovascular, anti-thrombotic, anti-aggregant, anti-adhesive, lipotropic properties as its activity on diastolic pressure, the association and the co-administration of at least a betaine with an anti-cholesterol agent allows improving the therapeutic effectiveness of such anti-cholesterol agents while reducing their side effects. Such a combination would be particularly advantageous compared to existing treatments as for protection, safety and increased convenience of use which it provides.

The compositions of the invention can be used to treat and/or prevent cardiovascular diseases, cerebrovascular diseases, occlusive cardiovascular diseases, vascular hypertension, lipidic metabolism disorders, hyperlipidaemia, cholesterols related disorders, triglycerides related disorders, lipoproteins related disorders, diabetes, type 2 diabetes, metabolic diseases, atherosclerosis, insulin resistance, insulin resistance syndrome, syndrome X and metabolic syndrome.

In one embodiment the combinations betaine/anti-cholesterol agents are particularly suitable to be used in methods of treatments of patients having and/or at risk to experience and/or to have one or more diseases and/or one or more conditions such as Parkinson Disease, Alzheimer Disease, diabetes, liver enzymes elevations, hyperhomocysteinemia, metabolic syndrome, obesity, hypercholesterolemia and triglycerides elevations.

The other aim of the present invention is a pharmaceutical composition, comprising a betaine and an anti-cholesterol agent, said composition allowing an excellent bioavaibility, as well as to assure in the preferred forms of realization an immediate liberation of one or more anti-cholesterol agent in the organism with a reduction, indeed a complete absence of secondary effects due to the anti-cholesterol agent.

The invention has equally for object a composition allowing obtaining an increased effectiveness of the anti-cholesterol agent, allowing thus to reduce the doses of anti-cholesterol agent.

For example, the compositions of the invention when placed in a capsule allow obtaining an improved dissolution curve in an aqueous environment exempt of tensioactive agents, other than betaine, comparatively with the product Tricor® (fenofibrate-Fournier).

In one embodiment the combinations betaine/fenofibrate are claimed to act synergistically to enhance nitric oxide production in a mammal and thus to have beneficial therapeutic effects in nitric oxide depletion and/or deficiency related pathologies.

### DESCRIPTION OF THE PREFERRED EMBODIMENTS

One of the manners for optimizing the invention consists to comprises adopting according to the physicochemical characteristics of each anti-cholesterol agents, claimed in the combination of the invention, the strategy of formulation which would optimize its therapeutic effectiveness, increase its security of use while reducing side effects and improving its form attractiveness in order to increase the observance of the treatments.

For example for the statins, in order to avoid washing, one can adopt formulations comprising compartments with different indices or curves of dissolutions which allow the absorption of a molecule followed by the absorption of a another, here preferably a statin then a betaine and optionally a third therapeutic agent.

For anti-cholesterol agents, preferably fibrates one can adopt micronization techniques where at least a betaine is used as tensioactive or surfactant, this allowing avoiding the use of others surfactants as the side effects linked to such surfactants. The invention has thus also as an aim a process of preparation of micronized fibrate (size average in weight of less 10 μm, in particular of less than 5 μm, preferably of less than 2 μm, even of less than 1 μm), in which solid fibrate is micronized in the presence of betaine, the ratio in weight fibrate/betaine being advantageously lower than 1.

In the case of the niacines one can realize the betaine/niacine mixture before adding the excipients necessary to the controlled release, which allows to reducing the amount of excipients. One can also chemically associate, synthesize or bind a betaine to a niacine, this allows to improving the bioavaibility, the pharmacokinetic profile and/or the pharmacological profile of at least one of the two substances. Thus the invention has also as an aim a niacine-betaine.

For the case of bile sequestrants, which when not being absorbed would thus increase the gastric residence time of at least a betaine in order to augment and/or to control its release, its bioavaibility and/or its absorption. For this purpose, at least a betaine can be bound, be associated and/or be synthesized with bile sequestrants.

When considering the association of molecules with sometimes antagonistic physicochemical characteristics, it will be in certain case useful to separately formulate each active ingredient of the combination of the invention and advantageously to isolate them one from the other.

For this purpose, for example, a capsule containing two compartments one containing the anti-cholesterol agent for an immediate release, the other containing at least a betaine being intended for a controlled release can be adopted. A solid or semi solid form containing betaine or anti-cholesterol can also be provided with one or several barriers or protective layers. In a particular aspect, the anti-cholesterol agent can be partly or completely released in controlled or delayed manner.

In another aspect, the capsule will comprise more than two compartments, said compartments having different dissolutions indices or curves and being able to release at least a betaine in a controlled manner and optionally at least an anti-cholesterol agent, said anti-cholesterol agent being optionally released in a controlled manner. In this case, optionally at least a compartment can contain a third therapeutic agent, for example aspirin.

In another aspect the anti-cholesterol agent and betaine and optionally other therapeutic agents could separated by one or more films and/or by one or more pharmaceutically acceptable excipients.

In another aspect the anti-cholesterol agent and the betaine can be partially or completely bound or associated.

The forms described in PCT/IB 2002/04923 (incorporated by reference herein) of the inventor can be used, but aspirin being replaced and/or supplemented by one or more anti-cholesterol agents, said agents being optionally suitable, to be micronized, to be put in micro and/or nano spheroid forms and/or to be spheronized in the presence of betaine and optionally in the presence of one or several excipients, said excipients being able optionally to ensure a controlled release of a betaine and/or an anti-cholesterol agent and/or to ensure an increased bioavaibility. In a particular aspect, the aforementioned forms will be able to comprise another additional therapeutic agent, preferably aspirin.

In another aspect of the invention, betaine could be used in combination with aspirin and an anti-cholesterol agent such as a statin for the manufacture of a drug. In this case betaine can be in controlled release or in immediate release.

In another aspect of the invention, betaine can be used as a surfactant in particular in the micronized, micro and/or nano spheroid and/or spheronized forms of the anti-cholesterol agents. Betaine as surfactant can be completely or partly in a form with immediate release and/or completely or partly in a form with controlled releases and/or liberations.

The advantage of using a betaine as surfactant or tensioactive in the combinations of the invention lies in the fact that betaine has weak toxicity contrarily to others surfactants used with anti-cholesterol agents such as notably sodium lauryl sulphate used in fenofibrate micronized formulations, notably in Tricor®. Betaine in this case can play the multiple role of surfactant or tensioactive, of therapeutic substance reducing the side effects of anti-cholesterol agents and of protective therapeutic substance in various pathologies in particular cardiovascular diseases and metabolic syndrome. Betaine is used also in this case to improve the bioavaibility of the anti-cholesterol agent comparatively to the bioavaibility of said agent without surfactant or tensioactive.

The composition according to the invention is thus advantageously free of tensioactive of the type of sodium lauryl sulphate. According to a particular form, the composition contains as surfactant or tensioactive, sensibly only betaine, in particular glycine betaine. The betaine, in particular glycine betaine, forms more than 90% in weight, preferably more than 95% in weight, more specifically more than 99% in weight of surfactants or tensioactives present in the composition.

Betaine, in particular glycine betaine, preferably in anhydrous form, as surfactant or tensioactive could be mixed with the agent anti-cholesterol before, during or after micronization and/or nanonization (reduction in particles smaller than micron). Betaine, in particular the glycine betaine, allows improving the anti-cholesterol agent bioavaibility, in particular fibrates and statins, for example when the anti-cholesterol agent is micronized or nanonized in the presence of betaine, in particular of glycine betaine. In a particular aspect, this mixture will additionally comprises excipients, notably excipients intended to ensure and/or to control a better bioavaibility, profile of release, time of gastric residence or absorption by the body, said excipients being suitable to be mixed before, during or after micronization.

The present invention also describes a kit comprising at least two formulations:

A first formulation for oral use or for oral administration comprising at least a betaine, its pharmaceutically acceptable salts, esters, precursors and their mixtures, and a second containing an anti-cholesterol agent, advantageously for oral use or for oral administration.

A kit comprising at least two formulations:

A first formulation for oral use or for oral administration comprising at least a betaine, its pharmaceutically acceptable salts, esters, precursors and their mixtures, said betaine being in a form with controlled and/or delayed and/or floating release, and a second containing an anti-cholesterol agent, advantageously for oral use or for oral administration.

In a particular aspect of the invention, bile sequestrants such as cholestyramines, colesevelam and colestipol can be associated with at least a betaine in order to increase gastric residence time of this betaine and to allow increasing its bioavaibility and/or to control its release and/or its absorption.

In another aspect of the invention betaines and anti-cholesterol agents can be formulated together in gels and/or suspensions said gels and/or suspensions optionally comprising solvents and/or excipients, said excipients increasing optionally the bioavaibility and/or controlling the release and/or the absorption of at least a betaine and/or at least an anti-cholesterol agent.

In a particular form betaines and/or anti-cholesterol agents can be pellicles covered and/or separated by one or more water-soluble and/or non water-soluble films.

The term betaine and/or at least a betaine and/or betaines in the field of the present invention refers to the betaines described and claimed in inventor's applications WO 00/51596, WO 02/062322 and PCT/IB 2002/04923, incorporated by reference herein. Betaines as well as the combinations of betaines between them, and the combinations of betaines with other molecules described in these applications, will be suitable in the field of the present invention to be formulated, manufactured, synthesized, combined and presented according to the invention in a floating form with controlled release or not and/or a controlled release form.

The terms “betaine” and/or “at least a betaine” and/or “betaines” employed in the present invention refer to the compounds chosen in the groups consisting of lipidic betaines, lipids of betaine, and/or betaines of formula (CH₃)₃N⁺—(CH₂)ₙ—COO⁻with n an integer from 1 to 5, (preferably the glycine betaine n=1), their pharmaceutically acceptable salts, their esters, their precursors and their mixtures. The terms “lipidic betaines” and “betaine lipids” refer to betaine lipids which are structural components of membranes commonly found in ferns, mosses, fungi, amoeba, eukaryotes such as nonseed plants and algae. Betaine lipids are ether-linked, nonphosphorous glycerolipids that resemble the more commonly known phosphatidylcholine in overall structure. Most common glycerolipids are containing a diacyl-glycerol moiety to which a polar head group is attached. This head group can be a carbohydrate moiety as in the very abundant plant galactolipids or a phosphorylester as in the glycerophospholipids, the most common lipid class in animals. Betaine lipids represent a third class of glycerolipids in which a quaternary amine alcohol is bound in an ether linkage to the diacylglycerol moiety. They can be obtained by extraction, by biosynthesis or by synthesis. The betaine lipid diacylglyceryl-O-4′-(N,N,-trimethyl)homoserine and a closely related isoform diacylglyceryl-O-2′-(hydroxymethyl)(N,N,-trimethyl)-β-alanine are the most common.

The terms “betaine being in a form with release controlled and/or delay and/or floating” refer and claim the forms and formulations described in inventor's WO 02/062322, PCT/IB 2002/04923 and BE 2003/0248, incorporated by reference herein. The aforementioned terms also refer to all the methods and known techniques for the skilled man, to control, maintain, delay, accelerate and/or increase absorption by the human body of one or more therapeutic substances as well as the combinations of these actions. These actions applying here preferably to betaines but can in an aspect of the invention also apply to anti-cholesterols.

In a particular aspect of the invention betaines in their controlled releases forms can be combined with other molecules, drugs and active ingredients known by the skilled man for their harmful effects on the liver and/or for raising transaminases and/or raising homocysteine. In such combinations, betaines will be able to reduce the side effects and/or to increase the therapeutic effect of said molecules.

Aspects and particularities of the invention are described in the claims.

The invention has thus as object a pharmaceutical composition for the treatment of cholesterol in human or to prevent cholesterol problems in human, said composition for oral use or for oral administration comprising:

(1) at least a betaine, its pharmaceutically acceptable salts, esters and precursors and theirs mixtures, said betaine being in a form with controlled and/or delayed and/or floating release and/or ready to be floating in contact with the gastric and/or intestinal medium and

(2) at least an anti-cholesterol agent chosen among the groups

a) of fibrates such as befibrates, bezafibrates, ciprofibrates, clofibrates, clofibrates calcium, aluminium clofibrates, pyridoxin clofibrates, clofibrides, fenofibrates, micronized fenofibrates, co-micronized fenofibrates, nanonized fenofibrates (reduced in particles of less than 1 μm), gemfibrozils and/or

b) of the statins group such as statins, such as atorvastatin calcium, cerivastatin, sodium, fluvastatin sodium, lovastatin, pravastatin sodium, simvastatin, Pitavastatin Rosuvastatin and their mixtures and/or

c) of the niacins group such as niacimides, inositol nicotinate, exaniacinate, sodium nicotinate and nicotinic acid and/or

d) of the group of bile sequestrants such as cholestyramines, colesevelam and colestipol.

e) mixtures of one or more compounds of these groups

Advantageously, betaine is with controlled release ensuring a release of betaine during at least 4 hours, advantageously during at least 6 hours, preferably during at least 12 hours.

Preferably, betaine is in a floating or ready to be floating form in the stomach and/or the intestine.

According to an advantageous embodiment, the composition is presented as unitary dose containing from 250 mg to 2500 mg of betaine, in particular from 400 mg to 1200 mg.

According to a detail of a preferred embodiment, the composition is presented as unitary dose containing an anti-cholesterol dose for anti-cholesterol treatment during 12 hours or at least 12 hours, in particular during 24 hours or at least 24 hours.

According to a characteristic of an embodiment, the composition comprises one or more solid or semi solid forms with controlled release of betaine not containing anti-cholesterol and one or more solid or semi solid forms containing an anti-cholesterol, but advantageously not betaine.

According to an advantageous detail, the pharmaceutical composition according to the invention contains a dose of an anti-cholesterol agent as to obtain a given or determined reduction of LDL, said dose corresponding to less than 80% in weight, advantageously with less than 60% in weight of the amount of said anti-cholesterol agent which administered alone allows a reduction in LDL equal to the said given or determined reduction in LDL.

According to a particular embodiment, the anti-cholesterol agent, in particular fenofibrate and/or a statin, are co-micronized or co-nanonized with betaine, in particular glycine betaine.

The invention has further as an aim a composition of fenofibrate containing fenofibrate co-micronized or reduced in particles of less than 1 μm (nanonized), in presence of betaine, in particular glycine betaine or a salt thereof. Preferably at least 50% of fenofibrate of the composition are co-micronized or reduced in particles of less than 1 μm m in presence of betaine.

The invention has as another aim a composition of statin containing a statin co-micronized or reduced in particles of less than 1 μm (nanonized) in presence of betaine, in particular glycine betaine or a salt thereof. Preferably at least 50% of the statin of the composition are co-micronized or reduced in particles of less than 1 μm in presence of betaine.

The invention has also for object a process of preparation and/or for obtaining a pharmaceutical composition for oral use.

The invention has thus for object a pharmaceutical composition for oral use comprising:

(1) at least a betaine compound chosen among the group constituted of pharmaceutically acceptable betaines, theirs pharmaceutically acceptable salts, and theirs mixtures, and

(2) at least an anti-cholesterol agent chosen among the groups constituted of:

a) pharmaceutically acceptable fibrates, such as befibrates, bezafibrates, ciprofibrates, clofibrates, clofibrates calcium, aluminum clofibrates, clofibrates of pyridoxine, clofibrides, fenofibrates, micronized fenofibrates, nanonized fenofibrates, gemfibrozils, and their mixtures,

b) pharmaceutically acceptable statins, such as atorvastatin calcium, cerivastatin, sodium, fluvastatin sodium, lovastatin, pravastatin sodium, simvastatin, Pitavastatin Rosuvastatin and their mixtures

said composition comprising at least one or more aforementioned anti-cholesterol agents, said one or more anti-cholesterol agents being at least partially dissolved in a phase containing at least the betaine compound and/or comprising one or more aforementioned anti-cholesterol agents forming at least a film recovering at least partially the betaine compound or a solid or semi-solid form containing the betaine compound and/or comprising at least one or more aforementioned anti-cholesterol agents forming at least a matrix in which is dispersed the betaine compound or a solid or semi-solid form containing the betaine compound.

Advantageously, the composition according to the invention is at least partially in a floating form or a form able to be floating in the stomach and/or the intestine. This allows then to combine an immediate release effect of the anti-cholesterol agent with an extended absorption of the betaine compound.

In an advantageous manner, the composition according to the invention is in a unitary dose form containing from 250 mg to 2500 mg, in particular from 400 mg to 1200 mg of betaine compound, and less than 300 mg of anti-cholesterol agent, advantageously less than 200 mg of anti-cholesterol agents, preferably less than 150 mg of anti-cholesterol agents, more particularly less than 120 mg of anti-cholesterol agents. The compositions according to the invention in advantageous realizations forms permit to reduce the amount of anti-cholesterol agent comparatively to the existing commercial compositions.

According to a realization form, the phase containing at least the betaine compound, in which the anti-cholesterol agent is partially dissolved, is in a solid or semi-solid form at a temperature of at least 30° C., in particular of at least 40° C., in preference of at least 55° C.

Preferably, the phase containing at least the betaine compound, in which the anti-cholesterol agent is partially dissolved, is in the form of tablets, mini-tablets and/or in the form of grains and/or particles such as spheres and/or spheroids and/or extrudats and/or marbles with a granulometry favorably inferior to 50 μm.

According to a particular realization form, the composition according to the invention comports:

(a) at least a phase containing at least the betaine compound, in which the anti-cholesterol agent is partially dissolved, and/or comprising one or more of said anti-cholesterol agents forming at least a film recovering at least partially the betaine compound or a solid or semi-solid form containing the betaine compound and/or comprising one or more anti-cholesterol agents forming at least a matrix in which the betaine compound or a solid or semi-solid form containing the betaine compound are dispersed, and

(b) at least a phase containing at least the betaine compound but exempt of anti-cholesterol agent.

This realization form is particularly advantageous when permitting the preparation of a combination betaine+anti-cholesterol compound for the preparation of dosage form containing different amounts of betaine.

Preferably, the phase containing at least the betaine compound, in which the anti-cholesterol agent is partially dissolved, and/or comprising one or more of said anti-cholesterol agents forming at least a film recovering at least partially the betaine compound or a solid or semi-solid form containing the betaine compound and/or comprising one or more anti-cholesterol agents forming at least a matrix in which the betaine compound or a solid or semi-solid form containing the betaine compound are dispersed, said phase being in a solid or semi-solid form at least partially covered by at least a phase exempt of anti-cholesterol agent, in particular covered by particles of betaine compound, said particles advantageously at least partially micronized or nanonized.

More particularly, the phase containing at least the betaine compound, in which the anti-cholesterol agent is partially dissolved, and/or comprising one or more of said anti-cholesterol agents forming at least a film recovering at least partially the betaine compound or a solid or semi-solid form containing the betaine compound and/or comprising one or more anti-cholesterol agents forming at least a matrix in which the betaine compound or a solid or semi-solid form containing the betaine compound are dispersed, said phase being in a solid or semi-solid form having a granulometry superior to 50 μm, and the phase exempt of anti-cholesterol agent being in the form of particles and/or in the form of layer with thickness or granulometry inferior to 50 μm, preferably inferior to 20 μm.

According to another form of realization, the composition possesses at least a core, possibly inert core, not containing any anti-cholesterol agent, and advantageously exempt of compound of betaine, the aforementioned core being covered by a phase containing at least the betaine compound in which the anti-cholesterol agent is dissolved and/or comprising at least a said anti-cholesterol agents forming at least a film recovering at least partially the betaine compound and/or a solid or semi-solid form containing the betaine compound and/or comprising one or more anti-cholesterol agents forming at least a matrix in which the betaine compound or a solid or semi-solid form containing the betaine compound are dispersed, said solid or semi-solid form having possibly an intermediate layer comprising at least a phase containing at least a betaine compound, said phase being exempt of anti-cholesterol agent. In one embodiment said core can contain one or more therapeutic agent different of betaines and anti-cholesterol agents, such as but not limited to aspirin, clopidogrel (Plavix®), fatty acids (such as Omega-3, Omega 5, Omega-6, Omega-7, or Omega-9 and the like), antidiabetic compounds such as one or more antidiabetic agents, including, but not limited to, sulfonylureas, biguanides, glitazones and other PPAR.gamma. agonists, PPAR.alpha. agonists, PPAR.delta. agonists, alpha.-glucosidase inhibitors, potassium channel antagonists, aldose reductase inhibitors, glucagon antagonists, activators of RXR, anti-obesity agents (5) and prodrugs thereof.

According to a detail of a realization form, the composition is in capsule form or in capsules containing the phase containing at least a betaine compound in which the anti-cholesterol agent is dissolved and/or comprising one or more aforementioned anti-cholesterol agents forming at least a film recovering at least partially the betaine compound or a solid or semi-solid form containing the betaine compound and/or comprising at least one or more aforementioned anti-cholesterol agents forming at least a matrix in which is dispersed the betaine compound or a solid or semi-solid form containing the betaine compound.

According to another detail of realization form, the phase containing at least a betaine compound, in which the anti-cholesterol agent is dissolved, is the product of a drying, at least partial, of an aqueous mixture of betaine compound and anti-cholesterol agent.

According to an advantageous detail, in particular when the combination betaine+anti-cholesterol agent presents is in the form of tablet, the composition contains at least a disintegration agent, in particular Explotab®.

In an advantageous manner, for the compositions according to the invention, the compound of betaine is the glycine betaine.

Preferably, the compositions according to the invention are in unitary dose forms containing an anti-cholesterol dose for anti-cholesterol treatment during 12 hours or at least 12 hours, in particular during 24 hours or at least 24 hours.

According to a detail of realization form, the composition comprises a betaine, aspirin and/or clopidogrel and an anti-cholesterol agent.

According to an advantageous detail of realization forms, the compositions according to the invention contain less than 1% in weight of tensioactive agent that is not a betaine compound.

In particular, the compositions according to the invention are exempt lauryl sulfate sodium.

According to a characteristic of realization forms of the compositions according to the invention, the ratio in weight betaine compound/anti-cholesterol agent of the phase containing at least a betaine compound in which the anti-cholesterol agent is dissolved and/or comprising one or more aforementioned anti-cholesterol agents forming at least a film recovering at least partially the betaine compound or a solid or semi-solid form containing the betaine compound and/or comprising at least one or more aforementioned anti-cholesterol agents forming at least a matrix in which is dispersed the betaine compound or a solid or semi-solid form containing the betaine compound, said ratio is superior to 1, advantageously superior to 2, preferably superior to 5, more preferably between 5 and 250 in particular comprised between 5 and 50.

Said ratio can be preferably comprised between 5 and 50 for fibrates, and preferably comprised between 10 and 500 for statins.

According to an advantageous detail of realization, the forms contain a dose of an anti-cholesterol agent to obtain a given or determined decrease of LDL, said dose corresponding to less than 80% in weight, advantageously with less than 60% in weight of the amount of said anti-cholesterol agent which administered alone allows a reduction in LDL equal to the said given or determined reduction in LDL.

According to another characteristic, the compositions according to the invention contain at least a tensioactive agent or mix constituted with more than 90% in weight, preferably with more than 95% in weight, more specifically with more than 99% in weight of one or more betaines, in particular of glycine betaine.

According to a special form, the anti-cholesterol agent, in particular fenofibrate is solubilized in presence of betaine.

According to a detail, the composition of fenofibrate contains equally fenofibrate co-micronized or reduced in particles of less than 20 μm to less than 1 μm, in presence of betaine, in particular of glycine betaine or of a salt thereof, and/or co-micronized statin or reduced in particles of less than 20 μm to less than 1 μm in presence of betaine, in particular of glycine betaine or of a salt thereof.

According to a particular detail, the composition comprises a phase containing a betaine in immediate release combined to an anti-cholesterol agent and a phase containing a betaine in a form with controlled and/or delayed release and/or floating form eventually combined to an anti-cholesterol agent.

The invention has also for object a preparation procedure of a composition according to any of the preceding claims and/or description and/or disclosures, in which:
one prepares one or more phases containing one or more anti-cholesterol agents in melted and/or dissolved forms, in which one or more betaine compounds are dispersed and/or at least partially put in solution; and
one operates one or more steps chosen among the group comprising heating step, cooling step and combinations thereof to obtain a solid and/or semi-solid composition containing the anti-cholesterol agent dissolved and/or comprising one or more of said anti-cholesterol agents forming at least a film recovering at least partially the betaine compound and/or a solid form semi-solid form containing the betaine compound and/or comprising one or more of said anti-cholesterol agents forming at least a matrix in which is dispersed the betaine compound and/or solid and/or semi-solid forms containing the betaine compound.

According to a process, Fenofibrate is carried in a water bath to its melting temperature (eventually in presence of a tensioactive and/or of a fusible polymer and/or of a fatty alcohol and/or of an oil with a melting point comprised between 50 and 100° C.) i.e. to a temperature of 79-82° C. or more (favorably less than 150° C.), before being mixed with betaine. In an embodiment one or more fatty acids chosen among the group consisting of Omega-3, Omega 5, Omega-6, Omega-7, or Omega-9, their esters, theirs pharmaceutical acceptable derivatives, theirs conjugations, theirs precursors, theirs salts and/or theirs combinations can be used.

In an aspect of the invention, the betaine can be dispersed as dry powder on fenofibrate in melting state. The ratio in weight of the mixture betaine/fenofibrate can vary from 1 to 50 or from 50 to 1, preferably from 1 to 25 or from 25 to 1, more preferably from 1 to 15 or from 15 to 1.

In a particular aspect the betaine can be reduced in particles with average size of less than 100 μm, of less than 50 μm, of less than 20 μm, of less than 10 μm, in particular of less than 5 μm, preferably of less than 2 μm, even of less than 1 μm. The mixture betaine/fenofibrate can be subjected to one or more steps of crushing and/or tabletting and/or micronization and/or nanonization and/or spheronization and/or trituration and/or mixing. In a particular aspect one or more of the steps of triturating and/or of mixing and/or of crushing and/and/or tabletting or of micronization and/or nanonization and/or spheronization and/or trituration can be realized according to different chronologies according to the needs of the invention.

In another aspect of the invention, the betaine compound can be solubilized in one or more pharmaceutically acceptable solvent, preferably water and/or alcohol, said solution been dispersed on fenofibrate in melting state. In a particular aspect the solution of betaine will be at saturation of betaine in solvent i.e. at maximum concentration of betaine soluble in solvent.

Nevertheless, various concentrations of betaine in the solution can be used, varying from 100% of said maximum concentration of betaine soluble in solvent to 0.1% of said maximum concentration of betaine soluble in solvent. The betaine solution, for the purpose to be dispersed on fenofibrate in melting state, can be reduced in droplets with average size of less than 100 μm, of less than 50 μm, of less than 20 μm, of less than 10 μm, in particular of less than 5 μm, preferably of less than 2 μm, even of less than 1 μm. In the mixture in solution the ratio in weight of betaine/fenofibrate can vary from 1 to 50 or from 50 to 1, preferably from 1 to 25 or from 25 to 1, more preferably from 1 to 15 or from 15 to 1.

After optionally one or more trituration steps and/or of mixing steps of the betaine and fenofibrate mixture solution, said mixture solution can be subjected one or more drying steps in order to withdraw, at least partially, preferably completely water and/or alcohol and/or pharmaceutically acceptable solvent and/or theirs mixtures.

The mixture betaine/fenofibrate, possibly completely dried, can be subjected further to one or more steps of crushing and/or tabletting and/or micronizations and/or nanonisations and/or spheronisations and/or triturations and/or mixing and/or dryings. In a particular aspect one or more steps of dryings and/or trituration and/or mixing and/or micronizations and/or nanonisations and/or spheronisations can be realized according to different chronologies and/or paths for the needs of the invention.

The betaine can be also dissolved in one or more others pharmaceutically acceptable solvent and/or their mixtures known by the man skilled in the art for the purposes of realizing the invention. In an embodiment, organic solvents (carbon-containing) may be preferred.

According to another process, fenofibrate in dry form, optionally micronized and/or nanonized, can be mixed with betaine in dry form optionally micronized. The ratio in weight of the mixture betaine/fenofibrate can vary from 1 to 50 or from 50 to 1, preferably from 1 to 25 or from 25 to 1, more preferably from 1 to 15 or from 15 to 1. In a particular aspect the betaine can be reduced in particles with average size of less than 100 μm, of less than 50 μm, of less than 20 μm, of less than 10 μm, in particular of less than 5 μm, preferably of less than 2 μm, even of less than 1 μm. The mixture betaine/fenofibrate can be subjected further to one or more steps of crushing and/or tabletting and/or micronizations and/or nanonisations and/or spheronisations and/or triturations and/or mixing and/or dryings. In a particular aspect one or more steps of dryings and/or trituration and/or mixing and/or micronizations and/or nanonisations and/or spheronisations can be realized according to different chronologies and/or paths for the needs of the invention.

According to another process, fenofibrate in dry form, optionally micronized can be dissolved in a solution of betaine. The betaine compound can be solubilized in one or more pharmaceutically acceptable solvent, preferably water and/or alcohol. In a particular aspect the solution of betaine will be at saturation of betaine in solvent i.e. at maximum concentration of betaine soluble in solvent.

Nevertheless, various concentrations of betaine in solution can be used, varying from 100% of said maximum concentration of betaine soluble in solvent to 0.1% of said maximum concentration of betaine soluble in solvent. In the mixture in solution the ratio in weight of betaine/fenofibrate can vary from 1 to 50 or from 50 to 1, preferably from 1 to 25 or from 25 to 1, more preferably from 1 to 15 or from 15 to 1.

After optionally one or more steps of triturating or of mixing the solution of betaine and fenofibrate, said mixed solution can be subjected to one or more drying steps in order to withdraw, at least partially, preferably completely, water and/or the solvents. The mixture betaine/fenofibrate can be subjected further to one or more steps of crushing and/or tabletting and/or micronizations and/or nanonisations and/or spheronisations and/or triturations and/or mixing and/or dryings. In a particular aspect one or more steps of dryings and/or trituration and/or mixing and/or micronizations and/or nanonisations and/or spheronisations can be realized according to different chronologies and/or paths for the needs of the invention.

The betaine can be also dissolved in one or more others pharmaceutically acceptable solvent and/or their mixtures known by the man skilled in the art for the purposes of realizing the invention. In an embodiment, organic solvents (carbon-containing) may be preferred.

In particular aspect of the invention, the combinations betaines/anti-cholesterol agents, preferably one or more fibrates and/or one or more statins, can be associated with one or more fatty acids chosen among the group consisting of Omega-3, Omega 5, Omega-6, Omega-7, or Omega-9, their esters, theirs pharmaceutical acceptable derivatives, theirs conjugations, theirs precursors, theirs salts and/or theirs combinations. The fatty acids can be associated before and/or during and/or after the combinations and/or processes of the invention.

For oral administration, the pharmaceutical compositions of the invention can take the form of solutions, suspensions, tablets, pills, capsules, powders, and the like. Tablets containing various excipients such as sodium citrate, calcium carbonate and calcium phosphate are employed along with various binders such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

In one embodiment, the combined compounds in the form of a powder, granules, micro-granules, micro-spheres, pellets and gels. The combined compounds can be in the form of a pharmaceutical unit dosage form said dosage form being selected from the group consisting of sachets, pouches, blisters and bags. Pharmaceutical unit dosage form of a composition containing at least a betaine, said dosage form being selected from the group consisting of sachets, pouches, blisters and bags, wherein the pharmaceutical unit dosage form is provided with moisture barrier property defined by an increase of weight of the composition of less than 1% after storage of the unit dosage form in sealed condition in an environment with a temperature of 38° C. and a relative humidity of 90% during 30 days.

Such individual sachet being possibly further submitted to an encrypting against counterfeiting and/or a notch to facilitate the tearing and/or the opening. As MVTR stands for “Moisture Vapor Transmission Rate”, a measure of the passage of gaseous H₂O through a barrier, the pharmaceutical oral unitary dose of betaine in a sealed dosage form from the group consisting of sachets, bags, blisters and pouches in which the dosage form is at least partly flexible, water impermeable and characterized by a protective barrier by a MVTR value inferior to 0.1 g/m² at 38 C. ° and 90% relative humidity during 24 hours.

In one embodiment, the sizes of the betaines particles can be selected so as to absorb minimally the water (for instance from micronized particles to an optimal size particles allowing a minimal water intake). Optionally the particles (or the dosage form such as a sugar-coated pill could be further enveloped by a surfactant having good moisture barrier) can be sugar-coated and optionally such particles can be trapped in a gel or a polymer before being packaged in a selected MVTR container or pharmaceutical unit dosage form.

For example the coating or primary packaging material could be a laminate which is made up of 12 μm PET, 25 μm Alufoil and a 50 μm PE inner heat-seal layer. Further high quality and clarity of surface decoration might be realized by gravure reverse printing process.

The complete barrier requirement for this highly hygroscopic product (betaine after being dried, i.e. its liquid content partially or completely removed) could be provided by a laminate of PET, PE and Alufoil. The single 250 to 5000 mg doses are easy to tear open and safe for mouth contact. In this dosage form betaine can be taken directly by mouth without the need to dissolve in water. Some flavoring agents might be added to mask betaines taste by the way augmenting patients' compliance. Geometrical forms which augment the facility of use can be privileged.

In one embodiment a stick format of the sachet will be preferred as it uses a minimum amount of material in relation to the volume of its contents and further by reducing the bag surface it allows also to reduce the MVTR.

The dosage forms can be optimized according to selected combinations of MVTR, betaine doses, tensile strengths, sizes, forms, coefficients of friction. The initial rate of moisture of the betaines can also be selected and/or controlled so as to lower the other parameters (MVTR, etc) thus augmenting the compliance of the dosage form. Betaine monohydrate and/or Betaine anhydrous and/or theirs mixtures solutions after being submitted to the processes of the invention can be dried and sachets as unit oral dosage forms of betaine(s) can be realized using as primary packaging material multilayer Alufoil material. The realized sachets will be weighted just after their manufacture and 1, 3, 6 and 12 months later, so as to determine the possible water intake of the betaine(s) inside the sealed dosage forms. The results could show that the weight variation is in accordance and in the limits allowed by the International Pharmacopoeia and come up to the FDA and/or BGA directives and the recommended and approved instructions given by EF for this kind (sachets/blister/pouches) of pharmaceutical dosage form. Due to the high hygroscopic properties of betaines, in one embodiment betaine monohydrate can be preferred. In effect the water intake of betaine monohydrate can be in a preliminary step controlled so as to avoid/control further moisture intake.

In one embodiment Betaine monohydrate and/or Betaine anhydrous and/or theirs mixtures are used “as is”, i.e. as provided in pharmaceutical grade (suitable for oral use) by the manufacturers after the sugar beet molasses separation processes or the chemical or biological synthesis. These betaines can be the packaged in such unit oral dosage form sachets having such moisture and/or oxygen and/or light barriers and/or tensile strength and/or coefficient of friction.

The coefficients of friction outside/outside and/or inside/inside will have to be carefully chosen so as to allow the maximum of the compound to be delivered at the administration. When absorbing moisture the betaine can start a process of higher size crystallization which can adhere inside the sachet making a part of the betaine unavailable upon administration. In the other side such medicament being destined to a daily utilisation during years, it is necessary to carefully choice the physical properties of the primary packaging material so as to have at the same time a good moisture barrier while having an easy opening, i.e. a “friendly” tensile strength allowing for instance elderly people to take easily their daily or twice daily medication. The primary packaging material must be easy to tear while possessing good moisture and/or oxygen barriers, such barriers preventing betaines deliquescence. Moisture sorption could lead to betaine particles agglomeration which can then adhere inside the sachet leading to a partial delivery of the drug after tearing. When carefully choose, selected and combined these parameters will allow compliance with the International Pharmacopoeia and Pharmaceutical Industry standards as they (parameters) will allow a better compliance of the end user, i.e. the patient. All the combinations do not work and only a careful selection of specific materials with particular parameters can provide this double compliance of sachet oral unit dosage forms of betaines.

Thus it is claimed here the combinations of the above physical characteristics of betaines forms (salts, sizes, coatings, polymers, etc) and the physical characteristics of the packaging materials (MVTR, tensile strength, coefficient of friction, tear strength, etc) which (the combinations) allow to augment the compliance of the pharmaceutical dosage form while retaining and respecting correct (according to international regulations and directives) conservation properties.

The invention will be described in greater detail by way of specific examples. The following examples are offered for illustrative purposes, and are not intended to limit the invention in any manner.

### EXAMPLE 1

Tablet of betaine fibrate

| Ingredient | mg/tablet |
|---|---|
| Betaine Anhydrous | 450.00 |
| Fenofibrate micronized | 100.00 |
| (5 to 20 μm) | |
| Lactose Ph. Eur. | 48.00 |
| Ethylcellulose | 110.00 |
| (Release .RTM.) | |
| Pure water pH. Eur. | 150.00* |
| Cetostearyl Alcohol | 42.00 |
| Ph. Eur. | |
| Magnesium Stearate | 20.00 |
| Ph. Eur. | |
| Talc Ph. Eur. | 30.00 |
| TOTAL | 800.00 |

*Eliminated during the process

Obtaining Process

Betaine, preferably anhydrous glycine betaine and lactose were shelled, then transferred in a vibrating granulator and vaporized with ethylcellulose and water.

The obtained grains are then dried at 60 degrees C. and passed through a 0.4 mm sieve. The granules obtained are then mixed with cetostearyl alcohol and the whole vigorously mixed. The obtained mixture is passed through a 0.5 mm sieve, let cooling, and then is mixed with the micronized fenofibrate, purified talc and magnesium stearate. The obtained result is compressed in a press in order to obtain tablets of 800 mg weight.

The obtained tablets are then covered by a film having the following characteristics:

| Ingredient | mg/tablet |
|---|---|
| Hydropropylmethylcellulose | 0.77 |
| Ph. Eur. 15 cps (Methocel E15) | |
| Hydroxypropylmethylcellulose | 3.87 |
| (Ph. Eur. 5 cps (Methocel E5) | |
| Opaspray M-1-7111B (33% solids) | 2.57 |
| Polyethylene glycol 400 USNF | 0.52 |
| Purified Talc Ph. Eur. | 0.27 |
| Purified water pH | 55.52* |

*Eliminated during the process

Example 1a: Example 1 is repeated in the same proportions, but with 350 mg anhydrous betaine and 200 mg fenofibrate.

### EXAMPLE 2

Tablet of betaine fibrate

| Ingredient | mg/tablet |
|---|---|
| Betaine Anhydrous | 350.00 |
| Fenofibrate micronized | 60.00 |
| (5 to 20 μm) | |
| Lactose Ph. Eur. | 35.00 |
| Ethylcellulose USNF | 90.00 |
| (Ethocel 45 CP) | |
| Pure water pH. Eur. | 120.00* |
| Cetostearyl Alcohol Ph. Eur. | 32.00 |
| Magnesium Stearate Ph. Eur. | 17.00 |
| Talc Ph. Eur. | 16.00 |
| TOTAL | 600.00 |

*Eliminated during the process

Obtaining Process

Betaine, preferably glycine betaine monohydrate, micronized fenofibrate and lactose were shelled, then transferred in a vibrating granulator and vaporized with ethylcellulose and water. The obtained grains are then dried at 60 degrees C. and passed through a 0.4 mm sieve. The granules obtained are then mixed with cetostearyl alcohol and the whole vigorously mixed. The obtained mixture is passed through a 0.5 sieve, let cooling, and then is mixed with purified talc and magnesium stearate. The obtained result is compressed in a press in order to obtain tablets of 600 mg weight.

The obtained tablets are then covered by a film having example 1 characteristics.

Example 2a: Example 2 is repeated in the same proportions, but with 290 mg anhydrous betaine and 120 mg fenofibrate.

### EXAMPLE 3

Tablet of Betaine Fibrate

Examples 1, 1a, 2 and 2a are repeated in the same proportions, but betaine and fenofibrate have preliminary be co-micronized with a tensioactive agent.

### EXAMPLE 4

Tablet of betaine niacin

| Ingredient | mg/tablet |
|---|---|
| Niacine | 455.00 |
| Betaine | 285.00 |
| Hydroxypropyl Methylcellulose 2910 | 45.00 |
| (Methocel E15LV, Dow) | |
| Hydroxypropyl Methylcellulose 2208 | 98.00 |
| (Methocel K100 mCR, Dow) | |
| Hydrogenated vegetable oil | 75.00 |
| (Lubritab, Mendell) | |
| Glyceryl Behenate 0.50 | 17.00 |
| (Compritol 888) | |
| Magnesium Stearate | 25.00 |
| TOTAL | 1000.00 |

Obtaining Process

To realize the tablet purified water is heated at 95 degrees C. in a stainless steel container. Methocel El 5LV in powder is slowly added while mixing until obtaining a homogeneous suspension. The rotation speed is adjusted in order to avoid excessive entry of air in the solution through the vortex. Then, very cold water is slowly added and the mixture vigorously mixed at a temperature lower than 20 degrees. C. until a clear solution is obtained. The mix is continued during 20 additional minutes. Hydrogenated vegetable oil passed then through a No 16 sieve and added in the mixer. Then, the powders of niacin and betaine monohydrate are added to the mixer and are mixed during about 15 minutes, then Methocel K100 mCR is added and the whole mixed again during 15 additional minutes. Then, solution of Methocel E 15LV is vaporized on the mixture which is further mixed during 2 minutes. The obtained dry pellets are passed through a No 16 sieve. The final result is compressed in an eccentric press in order to obtain 1000 mg tablets.

### EXAMPLE 5

Betaine Statin Capsule

Preparation of a betaine/statin pellet with prolonged release.

A mixture comprising 37% compressible Eudragit RPL RTM, 61% anhydrous betaine, 1% talc stearate and 1% magnesium is realized. After treatment in a mixer Turbula T2C, the mixture is compacted by means of an apparatus EKO Korsch with 40.00 N/cm² and is transformed into pellets by means of a Erweka TG II S granulator. The pellets were then sorted by means of a vibrating machine retaining only fractions having a diameter between 100 and 150 μm. The obtained pellets are used to fill multi-compartments capsules, whose compartment contains a statin (20 to 250 mg) intended to be released immediately in the gastro-intestinal tract, and the other compartment intended to contain the pellet of betaine (400 to 1000 mg) ready to form a floating form in the stomach or the beginning of the gastrointestinal tract.

Example 5 was operated with atorvastatin calcium, cerivastatin, sodium, fluvastatin sodium, lovastatin, pravastatin sodium, simvastatin, pitavastatin, rosuvastatin, and theirs mixtures.

### EXAMPLE 6

Example 5 was repeated but the statin was formulated or a form with controlled release, for example by means of polyacrylate (methacrylate) layer insoluble in water.

### EXAMPLE 7

Examples 5 and 6 were repeated, but instead of using a statin, each of the following compounds was respectively used: befibrate, bezafibrate, ciprofibrate, clofibrate, clofibrate calcium, aluminium clofibrate, pyridoxin clofibrate, clofibride, fenofibrate, micronized fenofibrate, gemfibrozil, cholestyramine, the colesevelam, the colestipol, niacimide, Pinositol nicotinate, the exaniacinate, the sodium nicotinate and nicotinic acid.

### EXAMPLE 8

Examples 1 and 2 were repeated, but using co-micronized fenofibrate in the presence of glycine betaine, the co-micronized fenofibrate having a size lower than 2 μm.

### EXAMPLE 9

Example 8 was repeated, but using nanonized fenofibrate (reduced in particles of size lower than 0.5 μm, in particular lower than 250 nm) in the presence of glycine betaine.

### EXAMPLE 10

Fenofibrate co-micronized with glycine betaine (granulometry size lower than 3 μm) is mixed to an aqueous solution containing 20% in weight of glycine betaine (80% water/20% glycine betaine).

The mixture is maintained under agitation for 10 minutes before being lyophilized as to obtain a dry product containing 15% in weight of fenofibrate and 85% in weight of glycine betaine. The product was grinded in a powder with granulometry size lower than 5 μm.

Gelatin capsules were filled with 500 mg powder (75 mg of fenofibrate) and 750 powder mg (112.5 mg of fenofibrate).

### EXAMPLES 11 AND 11a

The example 10 was repeated but fenofibrate amount was sufficient to obtain a powder containing 30% in weight of fenofibrate and 70% in weight of glycine betaine.

Gelatin capsules were filled with 300 mg powder and 400 mg powder (90 mg of fenofibrate and 120 mg of fenofibrate).

In the example 11a the powder contained 50% in weight of fenofibrate.

### EXAMPLE 12

Preparation of a betaine/fenofibrate pellet with prolonged release.

A mixture comprising 37% compressible Eudragit RPL RTM, 61% anhydrous betaine, 1% talc stearate and 1% magnesium is realized. After treatment in a mixer Turbula T2C, the mixture is compacted by means of an apparatus EKO Korsch with 40.00 N/cm² and is transformed into pellets by means of a Erweka TG II S granulator. The pellets were then sorted by means of a vibrating machine retaining only fractions having a diameter between 100 and 150 μm. The obtained pellets are used to fill multi-compartments capsules, whose compartment contains fenofibrate (200 mg or 100 mg of fenofibrate) intended to be released immediately in the gastro-intestinal tract, and the other compartment intended to contain the pellet of betaine (400 to 1000 mg) ready to form a floating form in the stomach or the beginning of the gastro-intestinal tract.

### EXAMPLES 13, 13a AND 13b

Sachet dosage form containing 3000 mg anhydrous betaine and 120 mg fenofibrate. The pharmaceutical unit dosage sachet is provided with moisture barrier property defined by an MVTR value inferior to 0.2 g/m², The coating or primary packaging used material was a laminate made up of 12 μm PET, 25 μm Alufoil and a 50 μm PE inner heat-seal layer.

Example 13a: Example 13 is repeated with 2000 mg betaine and 200 mg betaine/fenofibrate co-micronized (80% fenofibrate/20% betaine).

Example 13b: Example 13a is repeated but adding a viscosity agent

### EXAMPLE 14

Examples 13, is repeated but with one or more statins such as atorvastatin calcium, cerivastatin, sodium, fluvastatin sodium, lovastatin, pravastatin sodium, simvastatin, Pitavastatin Rosuvastatin, pharmaceutically acceptable salts thereof, and mixtures thereof. The statins being at a dose comprised between 10 and 150 mg and the betaine at a dose comprised between 250 and 5000 mg.

The embodiment described in the examples can be combined to realize the invention. The described examples illustrate but do not limit the invention.

What I claim is:

1. A pharmaceutical composition for oral administration comprising:
(1) at least a pharmaceutically acceptable betaine, its pharmaceutically acceptable salts, and their mixtures, said betaine and salts thereof being in a form selected from the group consisting of release controlled form, delay release form, floating form, forms ready to be floating in contact with the gastric medium, forms ready to be floating in contact with the intestinal medium, and combinations thereof, and
(2) at least an anti-cholesterol agent selected from the group consisting of:
A) pharmaceutically acceptable fibrates and pharmaceutically acceptable salts thereof;
B) pharmaceutically acceptable statins and pharmaceutically acceptable salts thereof;
C) pharmaceutically acceptable niacins and pharmaceutically acceptable salts thereof;
D) pharmaceutically acceptable bile sequestering agent and pharmaceutically acceptable salts thereof, and
E) mixtures of these.

2. The composition of claim 1, in which the pharmaceutically acceptable fibrate is selected from the group consisting of befibrates, bezafibrates, ciprofibrates, clofibrates, fenofibrate, pyridoxine, clofibrides, pharmaceutically acceptable salts thereof, and mixtures thereof.

3. The composition of claim 2, in which the pharmaceutically acceptable fibrate is selected from the group consisting of clofibrates, calcium clofibrates, aluminium clofibrates, pyridoxin clofibrates, clofibrides, fenofibrates, micronized fenofibrates, nanonized fenofibrates, gemfibrozils, and mixtures thereof.

4. The composition of claim 1, in which the pharmaceutically acceptable statin is selected from the group consisting of statins, such as atorvastatin calcium, cerivastatin, sodium, fluvastatin sodium, lovastatin, pravastatin sodium, simvastatin, Pitavastatin Rosuvastatin, pharmaceutically acceptable salts thereof, and mixtures thereof.

5. The composition of claim 1, in which the pharmaceutically acceptable niacin is selected from the group consisting of cholestyramines, colesevelam, colestipol, pharmaceutically acceptable salts thereof, and mixtures thereof.

6. The composition of claim 1, in which the betaine or the pharmaceutically acceptable salt thereof is in a controlled release form ensuring a release of betaine for at least 4 hours after the oral administration.

7. The composition of claim 1, in which the betaine or the pharmaceutically acceptable salt thereof is in a controlled release form ensuring a release of betaine for at least 6 hours after the oral administration.

8. The composition of claim 1, in which the betaine or the pharmaceutically acceptable salt thereof is in a controlled release form ensuring a release of betaine for at least 12 hours after the oral administration.

9. The composition of claim 1, in the form of unit dosage form comprising from 250 mg to 2500 mg of betaine.

10. The composition of claim 1, in the form of unit dosage form comprising from 400 mg to 1200 mg of betaine.

11. The composition of claim 9, in the form of a unit dosage form comprising a pharmaceutically effective amount of a anti-cholesterol agent for ensuring an anti-cholesterol treatment for about 12 hours.

12. The composition of claim 1, in the form of a unit dosage form comprising from 250 mg to 2500 mg of betaine and a pharmaceutically effective amount of a anti-cholesterol agent for ensuring an anti-cholesterol treatment for about 12 hours.

13. The composition of claim 1, in the form of a unit dosage form comprising from 250 mg to 2500 mg of betaine and a pharmaceutically effective amount of a anti-cholesterol agent for ensuring an anti-cholesterol treatment for about 24 hours.

14. The composition of claim 1, in the form of a unit dosage form comprising from 250 mg to 2500 mg of betaine and a pharmaceutically effective amount of a anti-cholesterol agent for ensuring an anti-cholesterol treatment for at least 12 hours.

15. The composition of claim 1, in the form of a unit dosage form comprising from 250 mg to 2500 mg of betaine, said unit dosage form comprising:
at least one solid or semi solid betaine controlled release form substantially free of said anti-cholesterol agent, and
at least one solid or semi-solid form comprising the anti cholesterol agent.

16. The composition of claim 1, in the form of a unit dosage form comprising from 250 mg to 2500 mg of betaine, said unit dosage form comprising:
at least one solid or semi solid betaine controlled release form substantially free of said anti-cholesterol agent, and
at least one solid or semi-solid form comprising the anti cholesterol agent, said form being substantially free of betaine.

17. The composition of claim 1, which further comprises a therapeutically acceptable amount of a compound selected from the group consisting of aspirin, di-aspirin, pharmaceutically acceptable salts thereof and mixtures thereof.

18. The composition of claim 1, which comprises a therapeutically effective amount of an anti-cholesterol agent for achieving a determined LDL reduction.

19. The composition of claim 1, which comprises a therapeutically effective amount of an anti-cholesterol agent for achieving a determined LDL reduction, whereby said therapeutically effective amount of the anti-cholesterol agent corresponds to less than 80% of the amount of said anti-cholesterol agent which when administered alone ensures said determined LDL reduction.

20. The composition of claim 1, which comprises a therapeutically effective amount of an anti-cholesterol agent for achieving a determined LDL reduction, whereby said therapeutically effective amount of the anti-cholesterol agent corresponds to less than 60% of the amount of said anti-cholesterol agent which when administered alone ensures said determined LDL reduction.

21. The composition of claim 1, which further comprises a surface active agent different from betaine and betaine salt, whereby the weight ratio betaine/said surface active agent different from betaine and betaine salt is greater than 10.

22. The composition of claim 1, which further comprises a surface active agent different from betaine and betaine salt, whereby the weight ratio betaine/said surface active agent different from betaine and betaine salt is greater than 20.

23. The composition of claim 1, which further comprises a surface active agent different from betaine and betaine salt, whereby the weight ratio betaine/said surface active agent different from betaine and betaine salt is greater than 100.

24. The composition of claim 1, which comprises a therapeutically effective amount of glycine betaine or a pharmaceutically acceptable salt thereof.

25. The composition of claim 1, in which the anti-cholesterol agent is in a form selected from the group consisting of fenofibrate co-micronized with betaine or a salt thereof, fenofibrate nanonized in presence of betaine or a salt thereof, fenofibrate dissolved in betaine or a salt thereof, and mixtures thereof.

26. The composition of claim 1, in which the anti-cholesterol agent is in a form selected from the group consisting of fenofibrate co-micronized with glycine betaine or a salt thereof, fenofibrate nanonized in presence of glycine betaine or a salt thereof, fenofibrate dissolved in glycine betaine or a salt thereof, and mixtures thereof.

27. The composition of claim 1, in which the anti-cholesterol agent is fenofibrate co-micronized with betaine or a salt thereof in particles of less than 1 μm.

28. The composition of claim 1, in which the anti-cholesterol agent is fenofibrate co-micronized with glycine betaine or a salt thereof in particles of less than 1 μm.

29. The composition of claim 1, in which the anti-cholesterol agent is fenofibrate with an weight average size of less than 1 μm.

30. A pharmaceutical composition for oral administration comprising:
(1) at least a pharmaceutically acceptable betaine, its pharmaceutically acceptable salts, and their mixtures, and
(2) at least an anti-cholesterol agent selected from the group consisting of:
A) pharmaceutically acceptable fibrates and pharmaceutically acceptable salts thereof;
B) pharmaceutically acceptable statins and pharmaceutically acceptable salts thereof; and
C) mixtures thereof,
whereby said composition comprises at least partly said anti-cholesterol agent in a form selected from the group consisting of anti-cholesterol agent at least partly dissolved into a phase comprising at least betaine, anti-cholesterol agent contained in a film coating particles comprising betaine, anti-cholesterol agent containing matrix in which betaine is dispersed, anti-cholesterol agent containing matrix comprising a dispersed solid or semi solid form containing betaine, and mixtures thereof.

31. The composition of claim 30, in a form selected from the group consisting of release controlled form, delay release form, floating form, forms ready to be floating in contact with the gastric medium, forms ready to be floating in contact with the intestinal medium, and combinations thereof.

32. The composition of claim 30, in which the pharmaceutically acceptable fibrate is selected from the group consisting of befibrates, bezafibrates, ciprofibrates, clofibrates, fenofibrate, pyridoxine, clofibrides, pharmaceutically acceptable salts thereof, and mixtures thereof.

33. The composition of claim 30, in which the pharmaceutically acceptable fibrate is selected from the group consisting of clofibrates, calcium clofibrates, aluminium clofibrates, pyridoxin clofibrates, clofibrides, fenofibrates, micronized fenofibrates, nanonized fenofibrates, gemfibrozils, and mixtures thereof.

34. The composition of claim 30, in which the pharmaceutically acceptable statin is selected from the group consisting of statins, such as atorvastatin calcium, cerivastatin, sodium, fluvastatin sodium, lovastatin, pravastatin sodium, simvastatin, Pitavastatin Rosuvastatin, pharmaceutically acceptable salts thereof, and mixtures thereof.

35. The composition of claim 30, in which the betaine or the pharmaceutically acceptable salt thereof is in a controlled release form ensuring a release of betaine for at least 4 hours after the oral administration.

36. The composition of claim 30, in which the betaine or the pharmaceutically acceptable salt thereof is in a controlled release form ensuring a release of betaine for at least 6 hours after the oral administration.

37. The composition of claim 30, in which the betaine or the pharmaceutically acceptable salt thereof is in a controlled release form ensuring a release of betaine for at least 12 hours after the oral administration.

38. The composition of claim 30, in the form of unit dosage form comprising from 250 mg to 2500 mg of betaine.

39. The composition of claim 30, in the form of unit dosage form comprising from 400 mg to 1200 mg of betaine.

40. The composition of claim 39, in the form of a unit dosage form comprising a pharmaceutically effective amount of a anti-cholesterol agent for ensuring an anti-cholesterol treatment for about 12 hours.

41. The composition of claim 30, in the form of a unit dosage form comprising from 250 mg to 2500 mg of betaine and a pharmaceutically effective amount of a anti-cholesterol agent for ensuring an anti-cholesterol treatment for about 12 hours.

42. The composition of claim 30, in the form of a unit dosage form comprising from 250 mg to 2500 mg of betaine and a pharmaceutically effective amount of a anti-cholesterol agent for ensuring an anti-cholesterol treatment for about 24 hours.

43. The composition of claim 30, in the form of a unit dosage form comprising from 250 mg to 2500 mg of betaine and a pharmaceutically effective amount of a anti-cholesterol agent for ensuring an anti-cholesterol treatment for at least 12 hours.

44. The composition of claim 30, in the form of a unit dosage form comprising from 250 mg to 2500 mg of betaine, said unit dosage form comprising:
at least one solid or semi solid betaine controlled release form substantially free of said anti-cholesterol agent, and
at least one solid or semi-solid form comprising the anti cholesterol agent.

45. The composition of claim 30, in the form of a unit dosage form comprising from 250 mg to 2500 mg of betaine, said unit dosage form comprising:
at least one solid or semi solid betaine controlled release form substantially free of said anti-cholesterol agent, and
at least one solid or semi-solid form comprising the anti cholesterol agent, said form being substantially free of betaine.

46. The composition of claim 30, comprising less than 300 mg of said anti-cholesterol agent.

47. The composition of claim 30, comprising less than 200 mg of said anti-cholesterol agent.

48. The composition of claim 30, comprising less than 150 mg of said anti-cholesterol agent.

49. The composition of claim 30, comprising less than 120 mg of said anti-cholesterol agent.

50. The composition of claim 30, which further comprises a therapeutically acceptable amount of a compound selected from the group consisting of aspirin, di-aspirin, pharmaceutically acceptable salts thereof and mixtures thereof.

51. The composition of claim 30, which comprises a therapeutically effective amount of an anti-cholesterol agent for achieving a determined LDL reduction.

52. The composition of claim 30, which comprises a therapeutically effective amount of an anti-cholesterol agent for achieving a determined LDL reduction, whereby said therapeutically effective amount of the anti-cholesterol agent corresponds to less than 80% of the amount of said anti-cholesterol agent which when administered alone ensures said determined LDL reduction.

53. The composition of claim 30, which comprises a therapeutically effective amount of an anti-cholesterol agent for achieving a determined LDL reduction, whereby said therapeutically effective amount of the anti-cholesterol agent corresponds to less than 60% of the amount of said anti-cholesterol agent which when administered alone ensures said determined LDL reduction.

54. The composition of claim 30, which further comprises a surface active agent different from betaine and betaine salt, whereby the weight ratio betaine/said surface active agent different from betaine and betaine salt is greater than 10.

55. The composition of claim 30, which further comprises a surface active agent different from betaine and betaine salt, whereby the weight ratio betaine/said surface active agent different from betaine and betaine salt is greater than 20.

56. The composition of claim 30, which further comprises a surface active agent different from betaine and betaine salt, whereby the weight ratio betaine/said surface active agent different from betaine and betaine salt is greater than 100.

57. The composition of claim 30, which comprises a therapeutically effective amount of glycine betaine or a pharmaceutically acceptable salt thereof.

58. The composition of claim 30, in which the anti-cholesterol agent is in a form selected from the group consisting of fenofibrate co-micronized with betaine or a salt thereof, fenofibrate nanonized in presence of betaine or a salt thereof, fenofibrate dissolved in betaine or a salt thereof, and mixtures thereof.

59. The composition of claim 30, in which the anti-cholesterol agent is in a form selected from the group consisting of fenofibrate co-micronized with glycine betaine or a salt thereof, fenofibrate nanonized in presence of glycine betaine or a salt thereof, fenofibrate dissolved in glycine betaine or a salt thereof, and mixtures thereof.

60. The composition of claim 30, in which the anti-cholesterol agent is fenofibrate co-micronized with betaine or a salt thereof in particles of less than 1 μm.

61. The composition of claim 30, in which the anti-cholesterol agent is fenofibrate co-micronized with glycine betaine or a salt thereof in particles of less than 1 μm.

62. The composition of claim 30, in which the anti-cholesterol agent is fenofibrate with an weight average size of less than 1 μm.

63. The composition of claim 30, comprising at least the anti-cholesterol agent at least partly dissolved in a betaine containing phase, whereby said betaine containing phase is solid or semi-solid at least at 30° C.

64. The composition of claim 30, comprising at least the anti-cholesterol agent at least partly dissolved in a betaine containing phase, whereby said betaine containing phase is solid or semi-solid at least at 40° C.

65. The composition of claim 30, comprising at least the anti-cholesterol agent at least partly dissolved in a betaine containing phase, whereby said betaine containing phase is solid or semi-solid at least at 55° C.

66. The composition of claim 63, in which the betaine containing phase has a form selected from the group consisting of tablets, mini tablets, spheroids, extrudats, and spheres.

67. The composition of claim 66, in which the betaine containing phase has a form selected from the group consisting of tablets, mini tablets, spheroids, extrudats, and spheres, said form having an weight average particle size of less than 50 μm.

68. The composition of claim 30, comprising:
(a) at least a first phase comprising said anti-cholesterol agent in a form selected from the group consisting of anti-cholesterol agent at least partly dissolved into a phase comprising at least betaine, anti-cholesterol agent contained in a film coating particles comprising betaine, anti-cholesterol agent containing matrix in which betaine is dispersed, anti-cholesterol agent containing matrix comprising a dispersed solid or semi solid form containing betaine, and mixtures thereof, and
(b) a second phase comprising betaine, but substantially free of said anti-cholesterol agent.

69. The composition of claim 30, comprising:
(a) at least a first phase comprising said anti-cholesterol agent in a form selected from the group consisting of anti-cholesterol agent at least partly dissolved into a phase comprising at least betaine, anti-cholesterol agent contained in a film coating particles comprising betaine, anti-cholesterol agent containing matrix in which betaine is dispersed, anti-cholesterol agent containing matrix comprising a dispersed solid or semi solid form containing betaine, and mixtures thereof, and
(b) a second phase comprising betaine, but substantially free of said anti-cholesterol agent, said second phase comprising particles selected from the group consisting of betaine containing particles with an weight average particle size of less than 50 μm and particles provided with a betaine containing layer with a thickness of less than 50 μm.

70. The composition of claim 30, comprising:
(a) at least a first phase comprising said anti-cholesterol agent in a form selected from the group consisting of anti-cholesterol agent at least partly dissolved into a phase comprising at least betaine, anti-cholesterol agent contained in a film coating particles comprising betaine, anti-cholesterol agent containing matrix in which betaine is dispersed, anti-cholesterol agent containing matrix comprising a dispersed solid or semi solid form containing betaine, and mixtures thereof, and
(b) a second phase comprising betaine, but substantially free of said anti-cholesterol agent, said second phase comprising particles selected from the group consisting of betaine containing particles with an weight average particle size of less than 20 μm and particles provided with a betaine containing layer with a thickness of less than 20 μm.

71. The composition of claim 30, the betaine containing phase in which the anti cholesterol agent is dissolved is prepared from the drying of an aqueous composition comprising betaine and the anti-cholesterol agent.

72. The composition of claim 30, which further comprises at least one disintegrating agent.

73. The composition of claim 30, which comprises less than 1% by weight of tensioactive agent different from betaine.

74. The composition of claim 30, which is free of sodium lauryl sulfate.

75. The composition of claim 30, in which the weight ratio betaine/anti-cholesterol agent is greater than 2.

76. The composition of claim 30, in which the weight ratio betaine/anti-cholesterol agent is greater than 5.

77. The composition of claim 30, in which the weight ratio betaine/anti-cholesterol agent is comprised between 5 and 50.

78. The composition of claim 30, which comprises:
(a) an immediate release form comprising betaine and the anti-cholesterol agent, and
(b) a betaine containing form selected from the group consisting of release controlled form, delay release form, floating form, forms ready to be floating in contact with the gastric medium, forms ready to be floating in contact with the intestinal medium, and combinations thereof.

79. A method of treatment of a patient suffering or at risk of suffering troubles related to cholesterol, in which said patient is administered a therapeutically effective amount of a pharmaceutical composition for oral administration comprising:
(1) at least a pharmaceutically acceptable betaine, its pharmaceutically acceptable salts, and their mixtures, said betaine and salts thereof being in a form selected from the group consisting of release controlled form, delay release form, floating form, forms ready to be floating in contact with the gastric medium, forms ready to be floating in contact with the intestinal medium, and combinations thereof, and
(2) at least an anti-cholesterol agent selected from the group consisting of:
A) pharmaceutically acceptable fibrates and pharmaceutically acceptable salts thereof,
B) pharmaceutically acceptable statins and pharmaceutically acceptable salts thereof,
C) pharmaceutically acceptable niacins and pharmaceutically acceptable salts thereof;
D) pharmaceutically acceptable bile sequestering agent and pharmaceutically acceptable salts thereof; and
E) mixtures of these.

80. A method of treatment of a patient suffering or at risk of suffering troubles related to cholesterol, in which said patient is administered a therapeutically effective amount of a pharmaceutical composition for oral administration comprising:
(1) at least a pharmaceutically acceptable betaine, its pharmaceutically acceptable salts, and their mixtures, and
(2) at least an anti-cholesterol agent selected from the group consisting of:
A) pharmaceutically acceptable fibrates and pharmaceutically acceptable salts thereof,
B) pharmaceutically acceptable statins and pharmaceutically acceptable salts thereof, and
C) mixtures thereof,
whereby said composition comprises at least partly said anti-cholesterol agent in a form selected from the group consisting of anti-cholesterol agent at least partly dissolved into a phase comprising at least betaine, anti-cholesterol agent contained in a film coating particles comprising betaine, anti-cholesterol agent containing matrix in which betaine is dispersed, anti-cholesterol agent containing matrix comprising a dispersed solid or semi solid form containing betaine, and mixtures thereof.
